(19) United States
(12) Patent Application Publication
(10) Pub. No.: US 2022/0011388 A1
(43) Pub. Date: Jan. 13, 2022

(54) METHODS TO PREDICT LIVER DISEASE MORTALITY USING LIPOPROTEIN LP-Z

(71) Applicants: LIPOSCIENCE, INC., Morrisville, NC (US); BETH ISRAEL DEACONESS MEDICAL CENTER, INC., Boston, MA (US)

(72) Inventors: Zhenghui Gordon Jiang, Winchester, MA (US); James D. Otvos, Cary, NC (US); Irina Shalaurova, Cary, NC (US); Elias J. Jeyarajah, Raleigh, NC (US); Margery A. Connelly, Rolesville, NC (US); Michael Curry, Needham, MA (US); Nezam Afdhal, Charleston, MA (US); Yury Popov, Brookline, MA (US); Maria Perez-Matos, Boston, MA (US)

(21) Appl. No.: 17/291,723

(22) PCT Filed: Nov. 7, 2019
(86) PCT No.: PCT/US2019/060290

§ 371 Date: May 6, 2021

Related U.S. Application Data
(60) Provisional application No. 62/757,505, filed on Nov. 8, 2018.

(51) Int. Cl. G01R 33/465; G01N 24/08; G01R 33/46

(52) U.S. Cl. CPC G01R 33/465; G01N 24/08; G01N 2800/52; G01N 2800/085; G01R 33/4625

(57) ABSTRACT

Described herein are methods for the determination of patient mortality from alcoholic hepatitis in biosamples by NMR spectroscopy and more specifically for the determination of a Z index score based on lipoprotein constituent LP-Z in blood plasma and serum.

### RELATED APPLICATIONS

This application claims the benefit of priority of U.S. Provisional Patent Application Ser. No. 62/757,505 filed Nov. 8, 2018, the contents of which are hereby incorporated by reference as if recited in full herein.

### FIELD

Described herein are methods and systems for the determination of constituents in blood plasma and serum and more specifically for the determination of lipoprotein constituents in blood plasma and serum.

### BACKGROUND

Alcoholic hepatitis (AH) is a common cause of inpatient admission for liver diseases in the United States. Among the spectrum of alcoholic liver diseases, AH causes the most acute presentation, with a mortality of 5-10% among all patients, and up to 30-50% in its severe form. Distinct from other forms of liver failure, the presentation of AH is hallmarked by a severe defect in blood clotting (coagulopathy) and stasis of the bile (cholestasis) that can occur in the absence of significant hepatocyte loss or advanced fibrosis. The mechanism for this profound hepatocellular dysfunction in severe AH remains poorly understood. Conventional AH treatment is limited to alcohol abstinence, nutritional support, and corticosteroids in selected patients for a potential short-term benefit. Liver transplantation may be possible for select AH patients. Disease risk stratification is a key challenge in the clinical management of AH and it remains difficult to predict outcome among patients with liver failure and select appropriate patient candidates for liver transplantation.

Several prognosticating strategies in AH have been studied, including Maddrey discriminant function (DF), Glasgow alcoholic hepatitis score (GAHS), age, serum bilirubin, INR and serum creatinine (ABIC) score, and Lillie model. However, these scores do not reliably predict mortality and guide clinical decisions regarding liver transplantation. Another scoring system used to assess the severity of chronic liver disease is the Model for End-Stage Liver Disease (MELD). MELD is a score calculated from serum creatinine, total bilirubin, the international normalized ratio (INR) of prothrombin time, and sodium concentration. MELD is generally a good predictor for 90-day mortality among patients with cirrhosis from various forms of chronic liver diseases, and is conventionally used to select patients for liver transplantation and to rank patients on liver transplant waiting lists.

Despite having a high MELD score, a significant proportion of patients with AH can recover with abstinence of alcohol and with supportive care, unlike patients with decompensated cirrhosis, where spontaneous recovery rarely occurs. A reliable prognosticative method could aid in identifying AH patients that would be candidates for organ transplantation.

One essential function of the liver is to regulate lipid and lipoprotein metabolism. The secretion of very low density lipoprotein particle (VLDL), a lipoprotein rich in triglyceride, is one way that a liver cell can export triglyceride accumulated inside the cell. VLDL metabolizes to low density lipoprotein (LDL), a particle rich in cholesterol ester. The conversion of VLDL to LDL in the circulation is dependent on a series of enzymes produced by the liver.

Recent data suggest that nuclear magnetic resonance (NMR) spectroscopy may be used to identify and quantify LDL and abnormal lipoproteins, including LP-X and LP-Z. By accurately determining the presence and quantity of lipoproteins in a biosample and associating the lipoprotein levels with patient outcomes, prognosticative methods can be improved, ultimately improving patient care. Therefore, methods and systems are needed for assays that accurately determine lipoproteins in a plasma or serum sample and predict patient mortality. Described herein are new methods and systems to accurately detect and quantify the amount of LP-Z in a biosample using NMR spectroscopy and correlate the amount of LP-Z to patient mortality.

### SUMMARY

Described herein are methods and systems to accurately determine the presence and amount of LP-Z in a biosample using NMR spectroscopy and generate a Z index score to predict patient mortality. The invention may be embodied in a variety of ways. In certain embodiments, methods and systems include determination of LP-Z in a subject or patient. In some embodiments, methods may predict a patient's response to therapy or a patient's likelihood of mortality within 90 days.

In some embodiments, a method of predicting mortality of a subject with AH comprises the steps of acquiring an NMR spectrum of a blood plasma or serum sample obtained from the subject and programmatically determining the presence of LP-Z and total apoB-containing lipoproteins in the sample based on the NMR spectrum of the sample. In some examples the NMR spectrum of the sample may include all subclasses of normal lipoproteins as well as abnormal lipoproteins LP-X, LP-Y, and LP-Z. In certain embodiments, the method further comprises calculating a Z index score. In some cases, a Z index greater than 0.6 may be associated with alcoholic hepatitis mortality in 90 days or less.

Yet other embodiments are directed to NMR analyzers. The NMR analyzer may include a NMR spectrometer, a probe in communication with the spectrometer, and a controller in communication with the spectrometer configured to obtain NMR signal of a defined single peak region of NMR spectra associated with LP-Z of a fluid specimen in the probe and generate a patient report providing a LP-Z level. In some examples, the probe may be a flow probe.

The controller can include or be in communication with at least one local or remote processor, wherein the at least one processor is configured to: (i) obtain a composite NMR spectrum of a fitting region of an in vitro plasma biosample; and (ii) deconvolve the composite NMR spectrum using a defined deconvolution model to generate the LP-Z level. In certain embodiments, the deconvolution model comprises at least one of high density lipoprotein (HDL) components, low density lipoprotein (LDL) components, VLDL (very low density lipoprotein)/chylomicron components, LP-X, and/or LP-Y and LP-Z.

Further features, advantages and details of the present invention will be appreciated by those of ordinary skill in the art from a reading of the figures and the detailed description of the preferred embodiments that follow, such description being merely illustrative of the present invention. Features described with respect with one embodiment can be incorporated with other embodiments although not specifically discussed therewith. That is, it is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. The foregoing and other aspects of the present invention are explained in detail in the specification set forth below.

### BRIEF DESCRIPTION OF FIGURES

The present disclosure may be better understood with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

FIG. 1 shows exemplary NMR spectra of human serum.

FIG. 2 shows VLDL, LDL or HDL subclasses of the exemplary NMR spectra.

FIG. 3 shows exemplary analysis of plasma using the LP-X deconvolution model that includes reference signals for LP-X and LP-Z.

FIG. 4 shows exemplary LP-Z concentrations in healthy patients and those with liver diseases as determined by NMR analysis.

FIG. 5 shows an exemplary Kaplan Meier Curve of Z index to predict 90 day survival in severe alcoholic hepatitis.

FIG. 6 shows exemplary repeated measurement of Z index to predict 90 day survival in severe alcoholic hepatitis.

FIG. 7 shows an exemplary lipoprotein profile in alcoholic hepatitis compared to a healthy subject.

FIG. 8 shows chemical structures of lipids and triglyceride.

FIG. 9 is a schematic showing lipoprotein metabolism in a healthy subject.

FIG. 10 shows an exemplary lipoprotein profile for LP-X and LP-Z in alcoholic hepatitis.

FIG. 11 is a schematic illustration of a system for analyzing a patient's risk using a Z index module and/or circuit using according to embodiments of the present invention.

### DETAILED DESCRIPTION

In the present application, the relationship between LP-Z as determined by NMR spectroscopy was explored for plasma samples from alcoholic hepatitis (AH) patients and the mortality of the AH patients was tracked. Described herein are new methods to accurately predict the mortality of an AH patient based on amount of LP-Z in a biosample using NMR spectroscopy. The invention may be embodied in a variety of ways.

In some embodiments, methods and systems include determination of LP-Z in a subject or patient. In some embodiments, methods may predict a patient's response to therapy or a patient's likelihood of mortality within 90 days.

In some embodiments, a method of predicting mortality of a subject with AH comprises the steps of acquiring an NMR spectrum of a blood plasma or serum sample obtained from the subject and programmatically determining the presence of LP-Z and apoB-containing lipoproteins in the sample based on the NMR spectrum of the sample, where the NMR spectrum of the sample includes LP-X and LP-Z. In some embodiments, the NMR spectrum of the sample further includes LP-Y. In certain embodiments, the method further comprises calculating a Z index score. In some cases, a Z index greater than 0.6 may be associated with AH mortality in 90 days or less.

Lipoprotein Z (LP-Z) is a low density lipoprotein (LDL)-like particle. As LDL, LP-Z carries one copy of apolipoprotein B (apoB) with amphipathic lipids on the surface and hydrophobic lipids in the core of the particle. The species referred to as LP-Z herein has previously been described as “highly triglyceride enriched LDL” (Kostner G M et al., Biochem J. 1976; 157: 401-407). Lipoprotein X (LP-X) is an abnormal multilamellar vesicular particle enriched in phospholipids and unesterified cholesterol that is quantifiable by nuclear magnetic resonance (NMR) spectroscopy. Conventional lipid panel may not detect the presence of LP-X or LP-Z.

### Terms and Definitions

Like numbers refer to like elements throughout. In the figures, the thickness of certain lines, layers, components, elements or features may be exaggerated for clarity. Broken lines illustrate optional features or operations unless specified otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about V.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The term “programmatically” means carried out using computer program and/or software, processor or ASIC directed operations. The term “electronic” and derivatives thereof refer to automated or semi-automated operations carried out using devices with electrical circuits and/or modules rather than via mental steps and typically refers to operations that are carried out programmatically. The terms “automated” and “automatic” means that the operations can be carried out with minimal or no manual labor or input. The term “semi-automated” refers to allowing operators some input or activation, but the calculations and signal acquisition as well as the calculation of the concentrations of the ionized constituent(s) is done electronically, typically programmatically, without requiring manual input. The term “about” refers to +/−10% (mean or average) of a specified value or number.

The term “biosample” refers to in vitro blood, plasma, serum, CSF, saliva, lavage, sputum, urine, or tissue samples of humans or animals. Embodiments of the invention may be particularly suitable for evaluating human blood plasma or serum biosamples. The blood plasma or serum samples may be fasting or non-fasting.

The term “patient” or “subject” is used broadly and refers to an individual that provides a biosample for testing or analysis.

The term “clinical disease state” means an at-risk medical condition that may indicate medical intervention, therapy, therapy adjustment or exclusion of a certain therapy (e.g., pharmaceutical drug) and/or monitoring is appropriate. Identification of a likelihood of a clinical disease state can allow a clinician to treat, delay or inhibit onset of the condition accordingly. Examples of clinical disease states include, but are not limited to, CHD, CVD, stroke, type 2 diabetes, prediabetes, dementia, Alzheimer's, cancer, arthritis, rheumatoid arthritis (RA), kidney disease, liver disease, pulmonary disease, COPD (chronic obstructive pulmonary disease), peripheral vascular disease, congestive heart failure, organ transplant response, and/or medical conditions associated with immune deficiency, abnormalities in biological functions in protein sorting, immune and receptor recognition, inflammation, pathogenicity, metastasis and other cellular processes.

### Methods to Measure LP-Z to Determine Z Index

Described herein are novel methods (i.e., assays) utilizing NMR to characterize LP-Z in a biological sample to diagnose or detect AH in a subject. In some embodiments, the method can predict mortality in AH patients. The methods may be embodied in a variety of ways.

NMR spectroscopy has been used to concurrently measure a full spectrum of circulating lipoproteins including very low density lipoprotein (VLDL), low density lipoprotein (LDL) and high density lipoprotein (HDL) particle subclasses from in vitro blood plasma or serum samples, as well as abnormal lipoprotein particles such as LP-X and LP-Z. See, U.S. Pat. Nos. 4,933,844, 6,617,167, U.S. patent application Ser. No. 16/188,435, filed Nov. 13, 2018, the contents of which are hereby incorporated by reference as if recited in full herein. In some embodiments, the sample can be blood, serum, plasma, cerebral spinal fluid, or urine.

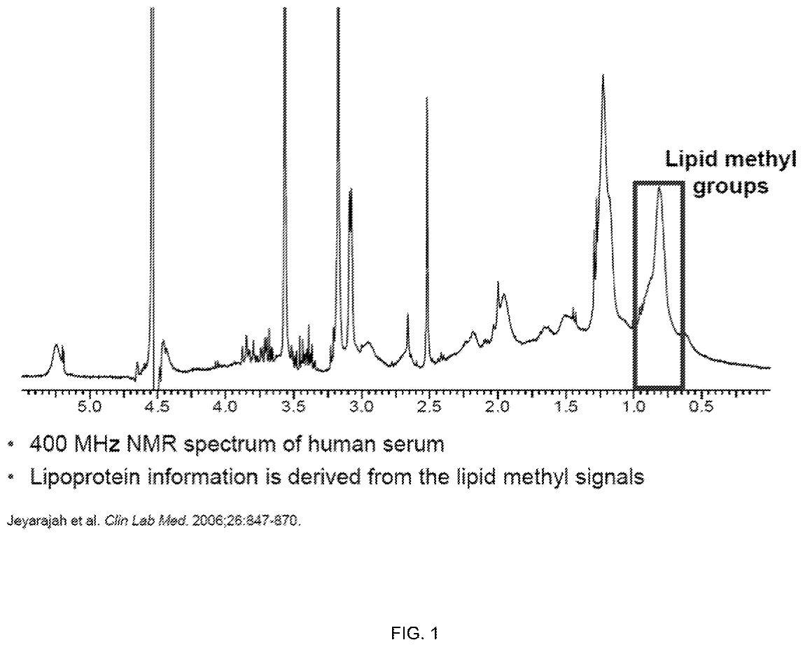

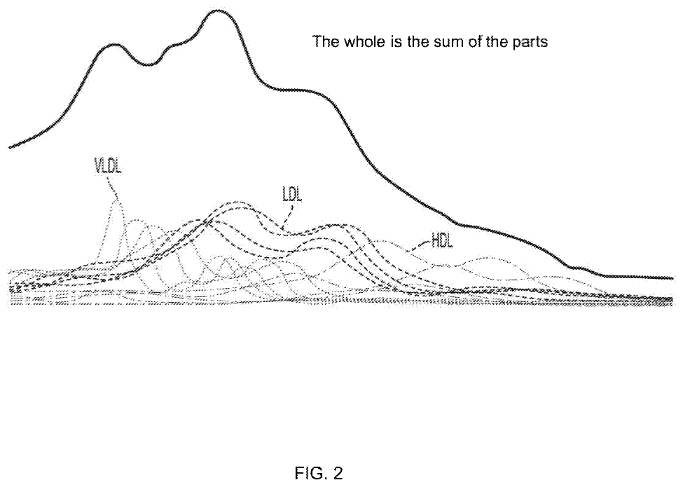

Generally stated, to evaluate the lipoproteins in a blood plasma and/or serum sample, the amplitudes of a plurality of NMR spectroscopy derived signals within a chemical shift region of NMR spectra are derived by deconvolution of the composite methyl signal envelope to yield subclass concentrations. FIG. 1 shows exemplary NMR spectra of human serum with the lipid methyl group highlighted. The subclasses are represented by many (typically over 60) discrete contributing subclass signals associated with NMR frequency and lipoprotein diameter. The NMR evaluations can decompose the measured plasma NMR signals to produce concentrations of different lipoprotein subpopulations, for VLDL, LDL and HDL. These sub-populations can be further characterized as associated with a particular size range within the VLDL, LDL or HDL subclasses as shown in FIG. 2, for example. As shown in FIG. 2, the subclass signals combine to produce the measured signal. The subclass signal amplitudes derived by deconvolution can provide concentrations for each subclass.

In the past, an “advanced” lipoprotein test panel, such as the NMR LIPOPROFILE® lipoprotein test, available from LapCorp, Burlington, N.C., has typically included a total HDL particle (HDL-P) measurement that sums the concentration of all the HDL subclasses and a total LDL particle (LDL-P) measurement that sums the concentration of all the LDL subclasses. The LDL-P numbers represent the concentration of those respective particles in concentration units such as nmol/L. The HDL-P numbers represent the concentration of those respective particles in concentration units such as μmon.

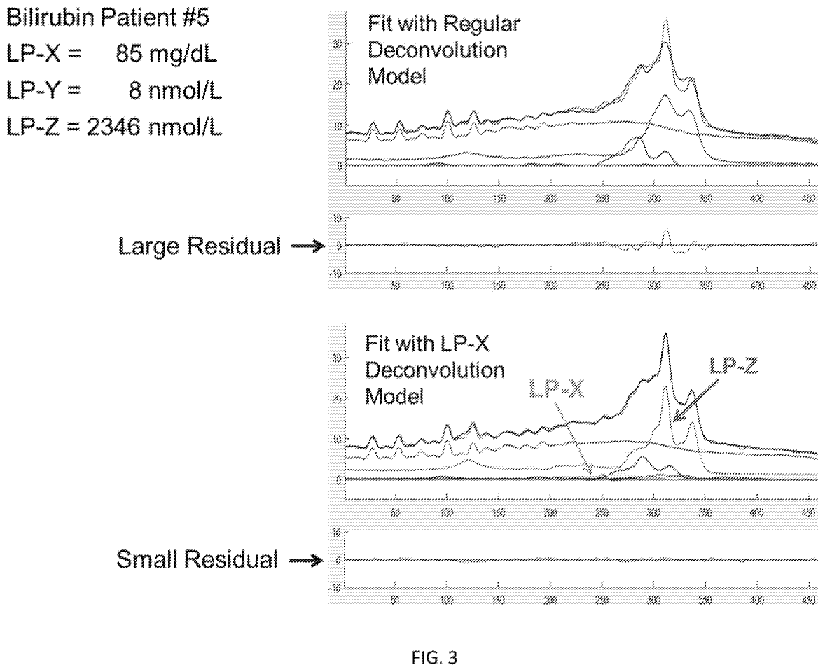

NMR analysis with refined deconvolution models has recently been used to determine concentration of LP-X and LP-Z in biosamples. FIG. 3 shows an example of the good fit and small residual signal resulting from analysis of plasma from a patient with high bilirubin when using the LP-X deconvolution model that includes reference signals for LP-X, LP-Y, and LP-Z.

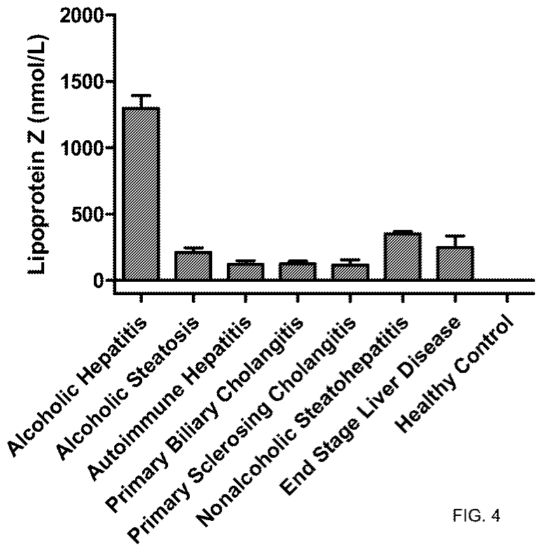

NMR spectroscopy may be used identify and quantify LP-Z in patients in whom LP-Z accumulates, such as those patients with alcoholic hepatitis (AH). As shown in FIG. 4, recent testing on plasma samples from AH patients utilizing an NMR-based methodology developed by LabCorp to quantify the profile of circulating lipoproteins in biosamples showed that exemplary patients with AH carry distinctively high levels of an abnormal lipoprotein LP-Z. In particular, the level of LP-Z may be distinctively high in patients with AH in comparison to healthy individuals (HC) or patients with other forms of chronic liver disease. For NMR to reliably be used for AH patient prognosis, the relationship of LP-Z determined by NMR and patient mortality must be understood.

The AH testing described above further identified that among AH patients, the levels of both LP-Z and total apoB-containing lipoprotein are inversely associated liver synthetic function, as measured by INR. While the levels of neither LP-Z nor total apoB-containing lipoprotein may be robustly associated with mortality in patients with AH, these two parameters can reciprocally predict mortality. LP-Z and total apoB-containing lipoprotein (VLDL, LDL, and LP-Z) can be used to predict mortality simultaneously. LP-Z may be positively associated with mortality, while total apoB-containing lipoprotein may be negatively associated with mortality. A novel biomarker, Z index, described herein, capitalizes on these associations with patient mortality. The Z index may be calculated by the following equation:

$$Z index = \frac{[ L P Z ]}{[ V L D L ] + [ L D L ] + [ L P Z ]}$$

where concentration units for the lipoprotein components are nmol/L.

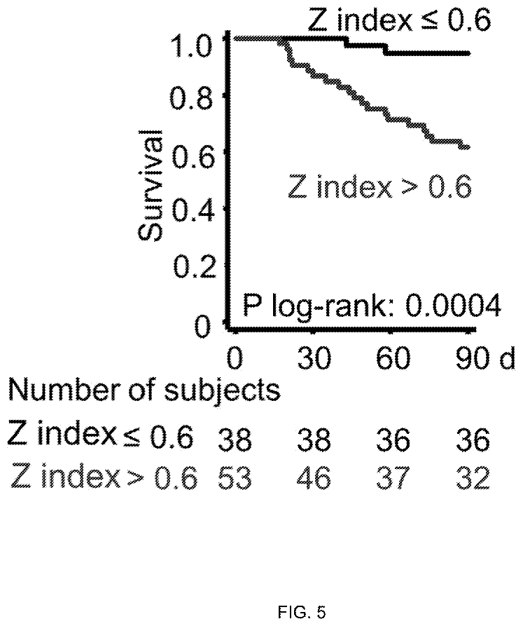

The Z index may represent the proportion of abnormal lipoprotein LP-Z in apoB-containing lipoproteins and may reflect the extent of liver impairment resulting in the derangement in circulating lipoproteins in AH. The Z index can be highly predictive of short-term mortality within 90 days. As shown in the exemplary Kaplan Meier Curve of FIG. 5, the Z index may be robustly associated with 90-day mortality. For every 1% increase in Z index, the hazard ratio of death increases 5% (95% CI 1.02-1.08, p=0.001). A threshold value for the Z index was determined to be 0.6. At a Z index less than 0.6, only about 5% of patients may die within 90 days of LP-Z identification (2 out of 38 test subjects in the data shown in FIG. 5). By contrast, nearly 40% of patients may be expected to die within 90 days of LP-Z identification when the Z index is greater than 0.6 (21 out of 53 test subjects died in 90 days in data shown in FIG. 5).

The Z index may be a more reliable predictor than MELD score, the current standard to prognosticate patient outcome with liver failure. As shown in Table 1, the Z index can significantly outperform MELD score in predicting 90-day mortality among patients with AH.

TABLE 1
Multivariate Cox proportional hazard regression

| Method | HR | 95% CI | P value |
|---|---|---|---|
| Z index (>0.6) | 8.4 | 1.9-36.4 | 0.004 |
| MELD | 1.0 | 0.9-1.2 | 0.5 |

The Z index may also be a more reliable predictor than other components in prognosticating outcome in AH as shown in Table 2.

TABLE 2
Confidence comparison for various strategies

| Z index | ≤0.6 | >0.6 | P value |
|---|---|---|---|
| Number | 38 | 53 | |
| INR | 2.0 ± 0.5 | 1.9 ± 0.4 | 0.4 |
| Bilirubin | 21.5 ± 9.3 | 25.3 ± 8.1 | 0.04 |
| Creatinine | 0.8 ± 0.5 | 1.2 ± 0.8 | 0.01 |

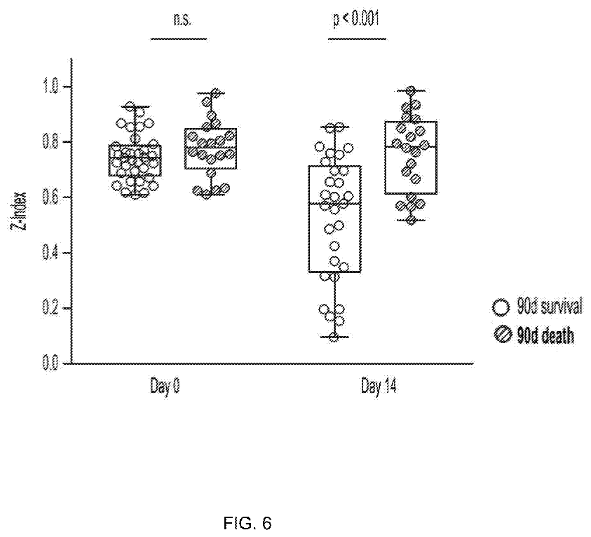

The Z index may be calculated using concentrations of LP-Z and total apoB-containing lipoproteins measured by NMR and may be used to effectively risk-stratify patients with severe AH. The effective risk-stratification may be particularly useful to help distinguish patients at low risk of death from those at high risk of death within 90 days. As shown in FIG. 6, for example, Z index can be used as a repeated measurement to predict outcome. The Z index among those that survived had declined by day 14 whereas the Z index for those who died remained steady.

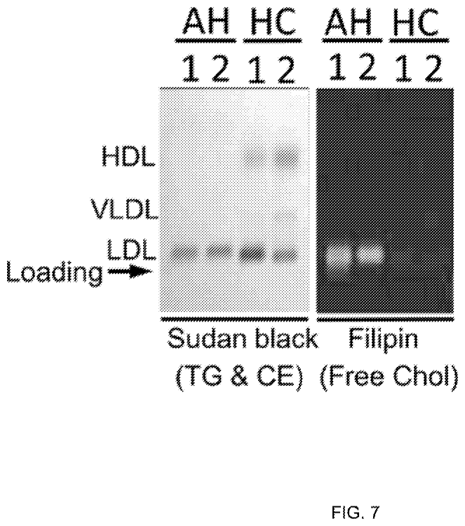

While the disclosure herein discloses LP-Z and apoB-containing lipoprotein via NMR spectroscopy, one skilled in the art understands that the Z index is not specific to NMR spectroscopy. For example, the concentration of LP-Z could be estimated using agarose gel electrophoresis coupled with lipid staining using Sudan black and Filipin. The concentration of apoB can be measured by ELISA. FIG. 7 shows that an exemplary lipoprotein profile in AH is distinctive as compared to that of an exemplary healthy subject (HC) in both Sudan black and Filipin tests.

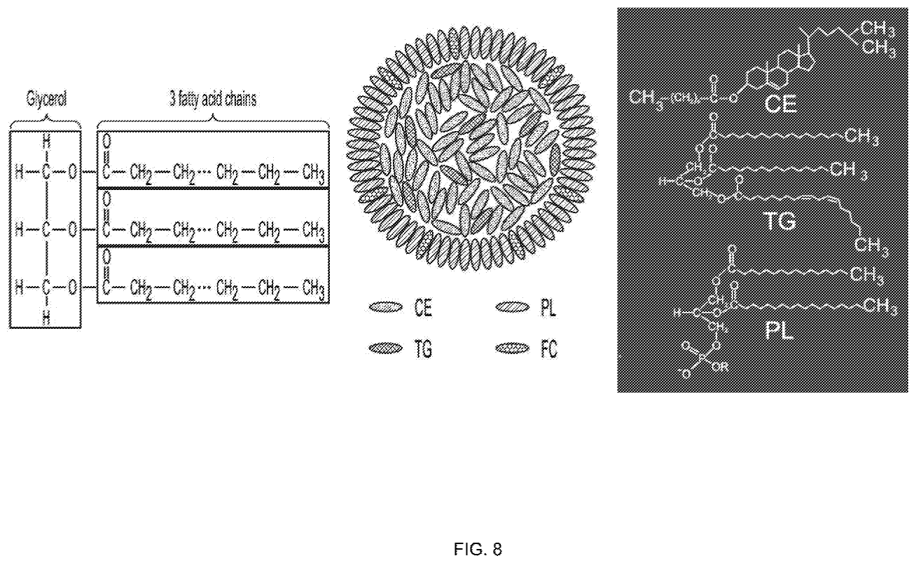

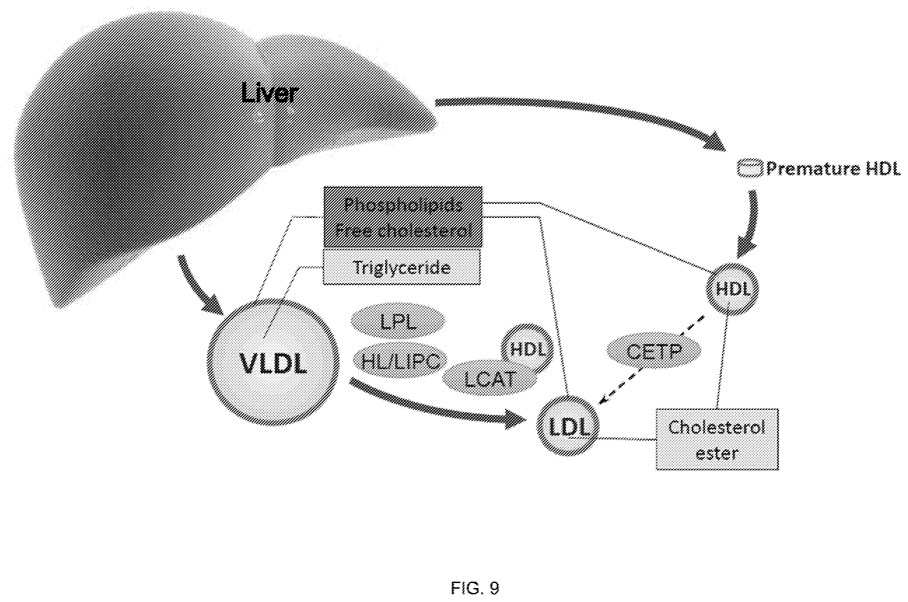

FIG. 8 shows a lipoprotein structure and chemical structures of phospholipid (PL), cholesterol ester (CE), and triglyceride (TG), and free cholesterol (FC). FIG. 9 shows the pathway of lipids in lipoprotein metabolism in a healthy subject. Most individuals (i.e. “normal” healthy subjects) have very low levels or no LP-X or LP-Z. In contrast, variable amounts of LP-Y are found in both healthy and diseased individuals. In subjects exhibiting the presence of LP-X or LP-Z, such as subjects having obstructive jaundice or AH, LP-Z levels may be elevated to varying degrees.

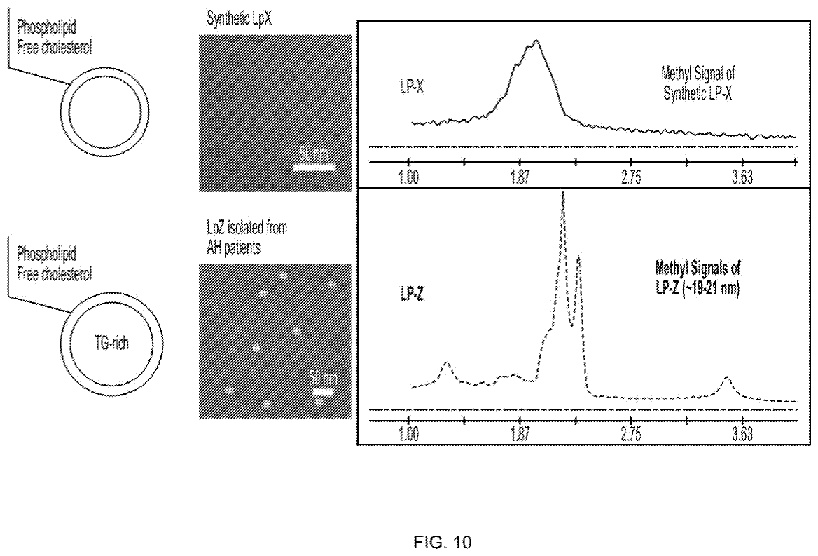

Methyl lipid signals from LP-X, LP-Y, and LP-Z each have a unique spectral shape and position in NMR spectroscopy, different from those of ‘normal’ lipoprotein particles. A unique pattern of circulating lipoprotein may be present in AH, characterized by the accumulation of abnormal lipoproteins LP-X and LP-Z. FIG. 10 shows an exemplary distinctive lipoprotein profile in AH patients. Elevated LP-X and LP-Z concentrations can distinguish healthy patients and those with liver diseases as determined by NMR analysis. These lipoproteins can be effective biomarkers for the risk stratification severe alcoholic hepatitis. The assays described herein utilize these unique spectral lineshapes to detect and quantify LP-X, LP-Y, and LP-Z in a serum or plasma sample.

In some embodiments, the method further comprises the step of producing a report listing the concentrations of the lipoprotein constituents present in the sample and likelihood of mortality. In some embodiments, a method of diagnosing a subject for the presence of LP-Z, comprises the steps of acquiring an NMR spectrum of a blood plasma or serum sample obtained from the subject and programmatically determining the presence of LP-Z in the sample based on the NMR spectrum of the sample, wherein the NMR spectrum of the sample includes LP-X, LP-Y, and LP-Z. In some embodiments, the acquiring step of the method comprises (a) producing a measured lipid signal lineshape for an NMR spectrum of a blood plasma or serum sample obtained from a subject; and (b) generating a calculated lineshape for the sample, the calculated lineshape being based on derived concentrations of lipoprotein components potentially present in the sample, wherein lipoprotein components include LP-X, LP-Y, and LP-Z, the derived concentration of each of the lipoprotein components being the function of a reference spectrum for that component and a calculated reference coefficient, wherein three of the lipoprotein components for which a concentration is calculated are LP-X, LP-Y, and LP-Z.

In some embodiments, the method further comprises (c) determining that the degree of correlation between the initial calculated lineshape of the sample and a measured lineshape of the sample; and (d) determining the presence of LP-Z based on the calculated lineshape if the degree of correlation between the calculated lineshape and the measured lineshape of the sample is above a predetermined threshold. In some embodiments, step (b) of the method comprises calculating the reference coefficients for the calculated lineshape based on a linear least squares fit technique. In some embodiments, the sample can be blood, serum, plasma, cerebral spinal fluid, or urine.

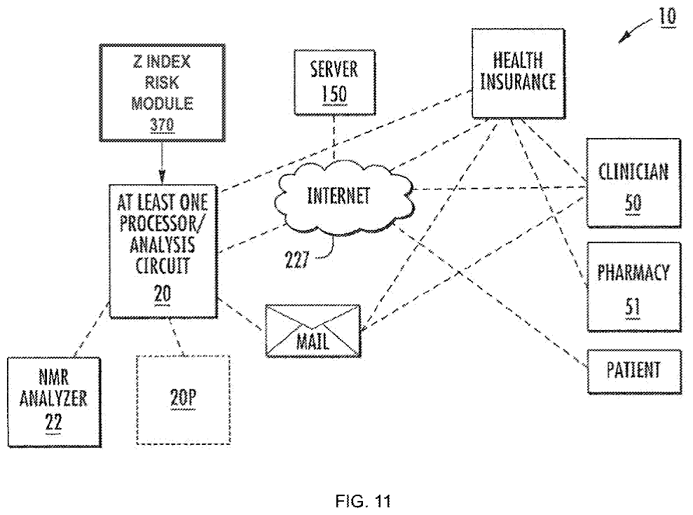

Referring now to FIG. 11, it is contemplated that most, if not all, the measurements can be carried out on or using a system 10 in communication with or at least partially onboard an NMR clinical analyzer 22 as described, for example, in U.S. Pat. No. 8,013,602, the contents of which are hereby incorporated by reference as if recited in full herein.

The system 10 can include a Z Index Risk Module 370 to collect data suitable for determining the Z index. The system 10 can include an analysis circuit 20 that includes at least one processor 20p that can be onboard the analyzer 22 or at least partially remote from the analyzer 22. If the latter, the Module 370 and/or circuit 20 can reside totally or partially on a server 150. The server 150 can be provided using cloud computing which includes the provision of computational resources on demand via a computer network. The resources can be embodied as various infrastructure services (e.g. computer, storage, etc.) as well as applications, databases, file services, email, etc. In the traditional model of computing, both data and software are typically fully contained on the user's computer; in cloud computing, the user's computer may contain little software or data (perhaps an operating system and/or web browser), and may serve as little more than a display terminal for processes occurring on a network of external computers. A cloud computing service (or an aggregation of multiple cloud resources) may be generally referred to as the “Cloud.” Cloud storage may include a model of networked computer data storage where data is stored on multiple virtual servers, rather than being hosted on one or more dedicated servers. Data transfer can be encrypted and can be done via the Internet using any appropriate firewalls to comply with industry or regulatory standards such as HIPAA. The term “HIPAA” refers to the United States laws defined by the Health Insurance Portability and Accountability Act. The patient data can include an accession number or identifier, gender, age and test data.

The results of the analysis can be transmitted via a computer network, such as the Internet, via email or the like to a patient, clinician site 50, to a health insurance agency 52 or a pharmacy 51. The results can be sent directly from the analysis site or may be sent indirectly. The results may be printed out and sent via conventional mail. This information can also be transmitted to pharmacies and/or medical insurance companies, or even patients that monitor for prescriptions or drug use that may result in an increased risk of an adverse event or to place a medical alert to prevent prescription of a contradicted pharmaceutical agent. The results can be sent to a patient via email to a “home” computer or to a pervasive computing device such as a smart phone or notepad and the like. The results can be as an email attachment of the overall report or as a text message alert, for example.

### Illustrative Embodiments of Methods, Systems, and Analyzers

As used below, any reference to a method, system, or analyzer is to be understood as a reference to each of those methods, systems, or analyzers disjunctively (e.g., “Illustrative embodiments 1-4” is to be understood as “Illustrative embodiment 1, 2, 3, or 4”).

Illustrative embodiment 1 is a method to predict patient mortality to alcoholic hepatitis comprising: acquiring an NMR spectrum of a biosample obtained from the subject; programmatically determining the concentration of LP-Z and total apoB-containing lipoproteins in the sample based on the NMR spectrum of the sample, wherein the NMR spectrum of the sample includes LP-X and LP-Z; and calculating a Z index score.

Illustrative embodiment 2 is the method of any preceding or subsequent embodiment, wherein the acquiring step of the method comprises: producing a measured lipid signal lineshape for an NMR spectrum of the biosample obtained from a subject; and generating a calculated lineshape for the sample.

Illustrative embodiment 3 is the method of any preceding or subsequent embodiment, wherein the calculated lineshape is based on derived concentrations of lipoprotein components comprising LP-X and LP-Z.

Illustrative embodiment 4 is the method of any preceding or subsequent embodiment, wherein the derived concentration of each of the lipoprotein components is a function of a reference spectrum for that component and a calculated reference coefficient.

Illustrative embodiment 5 is the method of any preceding or subsequent embodiment, wherein generating step comprises calculating the reference coefficients for the calculated lineshape based on a linear least squares fit technique.

Illustrative embodiment 6 is the method of any preceding or subsequent embodiment, further comprising: determining that the degree of correlation between the initial calculated lineshape of the sample and a measured lineshape of the sample; and determining the presence of LP-Z based on the calculated lineshape if the degree of correlation between the calculated lineshape and the measured lineshape of the sample is above a predetermined threshold.

Illustrative embodiment 7 is the method of any preceding or subsequent embodiment, wherein the Z index score comprises a concentration of lipoprotein LP-Z, LDL, and VLDL.

Illustrative embodiment 8 is the method of any preceding or subsequent embodiment, wherein the Z index is a ratio of LP-Z concentration to total apoB-containing lipoproteins concentration.

Illustrative embodiment 9 is the method of any preceding or subsequent embodiment, wherein the Z index is calculated by the following equation:

Z index=([LP-Z])/([VLDL]+[LDL]+[LP-Z]).

Illustrative embodiment 10 is the method of any preceding or subsequent embodiment, wherein a Z index of greater than 0.6 predicts patient mortality will occur in 90 days or less.

Illustrative embodiment 11 is the method of any preceding or subsequent embodiment, wherein the method predicts a likelihood of patient mortality within 90 days.

Illustrative embodiment 12 is the method of any preceding or subsequent embodiment, wherein the method predicts a likelihood of survival or patient response to treatment.

Illustrative embodiment 13 is the method of any preceding or subsequent embodiment, further comprising, before the programmatic determination, placing the sample of the subject in an NMR spectrometer; deconvolving the NMR spectrum; and calculating NMR derived measurements of a plurality of selected lipoprotein parameters based on the deconvolved NMR spectrum.

Illustrative embodiment 14 is the method of any preceding or subsequent embodiment, further comprising producing a report listing the concentrations of the lipoprotein constituents present in the sample and likelihood of mortality.

Illustrative embodiment 15 is the method of any preceding embodiment, wherein the biosample is one of blood, serum, plasma, cerebral spinal fluid, or urine.

Illustrative embodiment 16 is a NMR analyzer comprising: a NMR spectrometer; a probe in communication with the spectrometer; and a controller in communication with the spectrometer configured to obtain NMR signal of a defined single peak region of NMR spectra associated with LP-Z of a fluid specimen in the probe and generate a patient report providing a LP-Z level.

Illustrative embodiment 17 is the analyzer of any preceding or subsequent embodiment, wherein the controller is in communication with at least one local or remote processor, wherein the at least one processor is configured to: (i) obtain a composite NMR spectrum of a fitting region of the fluid specimen; and (ii) deconvolve the composite NMR spectrum using a defined deconvolution model to generate the LP-Z level.

Illustrative embodiment 18 is the analyzer of any preceding or subsequent embodiment, wherein the deconvolution model comprises at least one of high density lipoprotein (HDL) components, low density lipoprotein (LDL) components, VLDL (very low density lipoprotein)/chylomicron components, LP-X, LP-Y and LP-Z.

Illustrative embodiment 19 is the analyzer of any preceding or subsequent embodiment, wherein the probe is a flow probe.

Illustrative embodiment 20 is the analyzer of any preceding or subsequent embodiment, wherein the fluid specimen is an in vitro plasma biosample.

Illustrative embodiment 21 is the analyzer of any preceding embodiment, wherein the fluid specimen is a biosample of blood, serum, plasma, cerebral spinal fluid, or urine.

What is claimed is:

1. A method to predict patient mortality to alcoholic hepatitis comprising:
acquiring an NMR spectrum of a biosample obtained from the subject;
programmatically determining the concentration of LP-Z and total apoB-containing lipoproteins in the sample based on the NMR spectrum of the sample, wherein the NMR spectrum of the sample includes LP-X and LP-Z; and
calculating a Z index score.

2. The method of claim 1, wherein the acquiring step of the method comprises:
producing a measured lipid signal lineshape for an NMR spectrum of the biosample obtained from a subject; and
generating a calculated lineshape for the sample.

3. The method of claim 2, wherein the calculated lineshape is based on derived concentrations of lipoprotein components comprising LP-X and LP-Z.

4. The method of claim 3, wherein the derived concentration of each of the lipoprotein components is a function of a reference spectrum for that component and a calculated reference coefficient.

5. The method of claim 2, wherein generating step comprises calculating the reference coefficients for the calculated lineshape based on a linear least squares fit technique.

6. The method of claim 2, further comprising:
determining that the degree of correlation between the initial calculated lineshape of the sample and a measured lineshape of the sample; and
determining the presence of LP-Z based on the calculated lineshape if the degree of correlation between the calculated lineshape and the measured lineshape of the sample is above a predetermined threshold.

7. The method of claim 1, wherein the Z index score comprises a concentration of lipoprotein LP-Z, LDL, and VLDL.

8. The method of claim 1, wherein the Z index is a ratio of LP-Z concentration to total apoB-containing lipoproteins concentration.

9. The method of claim 1, wherein the Z index is calculated by the following equation:
Z index=([LP-Z])/([VLDL]+[LDL]+[LP-Z]).

10. The method of claim 1, wherein a Z index of greater than 0.6 predicts patient mortality will occur in 90 days or less.

11. The method of claim 1, wherein the method predicts a likelihood of patient mortality within 90 days.

12. The method of claim 1, wherein the method predicts a likelihood of survival or patient response to treatment.

13. The method of claim 1, further comprising, before the programmatic determination,
placing the sample of the subject in an NMR spectrometer;
deconvolving the NMR spectrum; and
calculating NMR derived measurements of a plurality of selected lipoprotein parameters based on the deconvolved NMR spectrum.

14. The method of claim 1, further comprising producing a report listing the concentrations of the lipoprotein constituents present in the sample and likelihood of mortality.

15. The method of claim 1, wherein the biosample is one of blood, serum, plasma, cerebral spinal fluid, or urine.

16. A NMR analyzer comprising:
a NMR spectrometer;
a probe in communication with the spectrometer; and
a controller in communication with the spectrometer configured to obtain NMR signal of a defined single peak region of NMR spectra associated with LP-Z of a fluid specimen in the probe and generate a patient report providing a LP-Z level.

17. The analyzer of claim 16, wherein the controller is in communication with at least one local or remote processor, wherein the at least one processor is configured to:
(i) obtain a composite NMR spectrum of a fitting region of the fluid specimen; and
(ii) deconvolve the composite NMR spectrum using a defined deconvolution model to generate the LP-Z level.

18. The analyzer of claim 17, wherein the deconvolution model comprises at least one of high density lipoprotein (HDL) components, low density lipoprotein (LDL) components, VLDL (very low density lipoprotein)/chylomicron components, LP-X, LP-Y and LP-Z.

19. The analyzer of claim 16, wherein the probe is a flow probe.

20. The analyzer of claim 16, wherein the fluid specimen is an in vitro plasma biosample.

21. The analyzer of claim 16, wherein the fluid specimen is a biosample of blood, serum, plasma, cerebral spinal fluid, or urine.
